(19) United States
(12) Patent Application Publication
(10) Pub. No.: US 2025/0032667 A1
(43) Pub. Date: Jan. 30, 2025

(54) BIOACTIVE CERAMICS CUSTOMIZED FOR TISSUE HEALING AND METHODS FOR PRODUCING SAME

(71) Applicant: Collagen Matrix, Inc., Oakland, NJ (US)

(72) Inventors: Hui-Chen Chen, Wayne, NJ (US); Shruti Saxena, Metuchen, NJ (US); Daniel Ammon, Webster, NY (US); Diana Legarda, Fair Lawn, NJ (US)

(73) Assignee: Collagen Matrix, Inc., Oakland, NJ (US)

(21) Appl. No.: 18/712,869

(22) PCT Filed: Nov. 29, 2022
(86) PCT No.: PCT/US2022/051231

§ 371 Date: May 23, 2024

Related U.S. Application Data
(60) Provisional application No. 63/283,612, filed on Nov. 29, 2021.

(51) Int. Cl. A61L 27/10; A61L 27/12; A61L 27/54; A61L 27/56

(52) U.S. Cl. CPC A61L 27/10; A61L 27/12; A61L 27/54; A61L 27/56; A61L 2300/102; A61L 2300/112; A61L 2430/02; A61L 2430/10

(57) ABSTRACT

Bioactive ceramic particles (such as bioglass) that are customized with ions to enhance tissue healing. The ions may include one or more of Magnesium, Copper, Cobalt, Silver, Aluminum, Iron, Manganese, Zinc, Calcium, Lithium, Gallium, Strontium and/or other Group 5 based ions. Medical applications for use of the customized bioglass particles include wound (dermis) repair, orthopedics (bone), spine, tendon, ligaments, cartilage, neurologic and dental. Embodiments of the customized bioglass particles contain multiple layers, each layer having a different composition. In some embodiments, the ions in each layer work in conjunction with a biologic process. Also disclosed are methods for forming the customized bioglass particles.

### RELATED APPLICATION

This application claims priority to U.S. Provisional Patent Application Ser. No. 63/283,612, filed Nov. 29, 2021, the disclosure of which is hereby incorporated by reference in its entirety.

### FIELD OF THE INVENTION

Embodiments of the invention pertain to bioactive glass ceramics (e.g., bioglass) that are customized to match the natural healing process of bone, or other tissues, and methods of making such bioactive glass ceramics.

### BACKGROUND OF THE INVENTION

Bioactive glass ceramics are highly reactive surfaces formed by melt or sol-gel techniques. Bioactive glass forms a hydroxy-carbonated apatite layer when immersed in biological fluid, which enhances protein adsorption to the surface of the bioactive glass implant and integration with surrounding bone. The rate of ion release from the bioglass surface is determined by the Ca:P ratio, composition, and microstructure. The initial reaction of some bioactive glasses with biological fluids causes local pH to increase; some studies propose that this is beneficial to cell activity and hydroxyapatite (HA) production.

The 45S5 form of bioglass has been used for various medical applications, including bone defects, restorative dentistry, and craniofacial surgery. In vitro assays comparing osteoblast response to different bioglass formulations indicate that the phosphate content is an important variable. Lower phosphate content, typical of the 45S5 bioglass that is used clinically, supports cell attachment and osteoblastic differentiation, whereas other forms of bioglass are less effective.

Bioactive glasses are composed of SiO2, CaO2, P2Os, and Na2O, but in proportions that are different from stable soda-lime silica glasses used in non-biologic applications. The glasses can be converted into a glass-ceramic by the addition or formation of crystals within the glass itself Both bioglasses and bioactive glass-ceramics have surfaces on which hydroxycarbonate apatite (HCA) precipitates and crystallizes within an hour of implantation.

Other mechanisms of activity that enable bioactive glasses to act as materials for bone repair have been investigated since the first work of Hench et al. at the University of Florida. Early attention was paid to changes in the bioactive glass surface. Five inorganic reaction stages are commonly thought to occur when a bioactive glass is immersed in a physiological environment, as shown in FIG. 1A These stages include:
1. Ion exchange in which modifier cations (mostly Na+) in the glass exchange with hydronium ions in the external solution.
2. Hydrolysis in which Si—O—Si bridges are broken, forming Si—OH silanol groups, and the glass network is disrupted.
3. Condensation of silanols in which the disrupted glass network changes its morphology to form a gel-like surface layer, depleted in sodium and calcium ions.
4. Precipitation in which an amorphous calcium phosphate layer is deposited on the gel.
5. Mineralization in which the calcium phosphate layer gradually transforms into crystalline hydroxyapatite, that mimics the mineral phase naturally contained with vertebrate bones.

Later, it was discovered that the morphology of the gel surface layer was a key component in determining the bioactive response. This was supported by studies on bioactive glasses derived from sol-gel processing. Such glasses could contain significantly higher concentrations of SiO2 than traditional melt-derived bioactive glasses and still maintain bioactivity (i.e., the ability to form a mineralized hydroxyapatite layer on the surface). The inherent porosity of the sol-gel-derived material was cited as a possible explanation for why bioactivity was retained, and often enhanced with respect to the melt-derived glass.

The HCA crystals provide a base for adsorption of components of the extracellular matrix and subsequent interactions with cells. The biologic response to bioactive glasses and glass-ceramics involves attachment of bone; however, interactions between the bioglasses and bone are restricted to the surface. The relative bioactivity of the bioglasses for bone and for soft tissue relates to the composition of the bioglasses, which is a major determinant of their bioactivity. Thus, the relative selectivity of bonding between bioglasses and bone versus soft tissue can be manipulated by alterations in their composition.

### SUMMARY OF THE INVENTION

Provided herein is a method for forming a bioglass composition containing ions for targeted application

Also provided herein are bioglasses comprising Magnesium, Copper, Cobalt, Silver, Aluminum, Iron, Manganese, Zinc, Calcium, Lithium, Gallium, Strontium and/or other Group 5 based ions.

Further provided herein are bioglasses having composition ions in a concentration range of 0.1% to 30% by weight.

Further provided herein are bioactive ceramic particles, each of the particles comprising a least one ion.

Further provided herein are applications for use of the bioglasses that include wound (dermis) repair, orthopedics (bone), spine, tendon, ligaments, cartilage, neurologic and dental.

Further provided herein is a method for forming a bioglass containing layers, where each layer has a different composition. In some embodiments, the ions in each layer work in conjunction with a biologic process.

Further provided herein is a layered bioglass particles where the ions in each layer are time released.

Further provided herein is a layered bioglass particle where the ions in each layer work in conjunction with a biologic process.

Further provided herein is a method for forming bioglass compositions containing layers, where at least one layer is a calcium phosphate layer.

Also provided herein is a method for forming a medical device comprising the above bioglasses as well as calcium phosphate, collagen, gelatin, glycosaminoglycans (GAGs), peptides, growth factors, synthetic polymers, sugars, and/or synthetic derivatives of collagen and gelatin and peptides and GAGs. In some embodiments, the device is produced by molding, subtractive manufacturing (milling) or additive manufacturing (3D printing).

Further provided herein are bioactive ceramic particles, each of the particles comprising a plurality of layers, each layer arranged to include a group of ions active in a separate stage of bone healing.

Further provided herein is a sol-gel synthesis method for forming bioactive ceramic particles each having a plurality of layers including a group of ions active in a separate stage of bone healing, the method comprising:
a. placing one or more precursors in a container to form a precursor solution;
b. adding one or more ions to the precursor solution;
c. mixing the ion/precursor solution;
d. subjecting the resulting mixture to gelation at room temperature;
e. aging the resulting gel in an oven;
f. drying the aged gel in an oven;
g. subjecting the dried gel to calcination to form the bioactive ceramic;
h. milling the resulting customized bioactive ceramic into a powder; and
i. sieving the powder to obtain particles having one or more specific particle sizes.

Further provided herein is a melt-derived synthesis method for forming bioactive ceramic particles each having a plurality of layers including a group of ions active in a separate stage of bone healing, the method comprising:
a. loading one or more precursors in an agate mortar;
b. melting the loaded agate mortar in a furnace;
c. quenching the melted agate mortar in distilled water at room temperature to obtain a customized bioactive ceramic frit;
d. drying the resulting customized bioactive ceramic frit in an over;
e. milling the dried customized bioactive ceramic into a powder; and
f. sieving the powder to obtain particles having one or more specific particle sizes.

### BRIEF DESCRIPTION OF THE DRAWINGS

Embodiments of the invention are further described but are in no way limited by the following illustrations.

FIG. 1A is a schematic illustration of a step-by-step integration of bioactive glass with bone;

FIG. 1B is a schematic illustration of the timeline of the bone healing process following fracture;

FIG. 2 is a schematic view of the bone healing process and ions involved in same;

FIG. 3 is a is a schematic view of a spherical bioglass sphere with multiple layers, according to an embodiment of the present invention;

FIG. 4 is a flow chart showing a method of forming customized bioglass particles according to an embodiment of the present invention;

FIG. 5 is a flow chart showing a method of forming customized bioglass particles according to another embodiment of the present invention;

FIG. 6 is a schematic view of an alternate embodiment of the present invention in coordination with the bone healing process;

FIG. 7 is a schematic view of the cellular assays used to evaluate the therapeutic effects of customized bioglass particles according to various embodiments of the present invention;

FIG. 8 is a graph showing vascular endothelial growth factor (VEGF) gene expression levels in mesenchymal stem cells exposed to the ionic dissolution products from customized bioglass particles according to various embodiments of the present invention;

FIG. 9 shows Alizarin red staining demonstrating mineralization levels for mesenchymal stem cells exposed to the ionic dissolution products from customized bioglass particles according to various embodiments of the present invention;

FIG. 10A is a graph showing osteopontin (SPPI) gene express10n levels in mesenchymal stem cells exposed to the ionic dissolution products from customized bioglass particles according to various embodiments of the present invention;

FIG. 10B is a graph showing osteocalcin (BGLAP) gene expression levels in mesenchymal stem cells exposed to the ionic dissolution products from customized bioglass particles according to various embodiments of the present invention;

FIG. 10C is a graph showing bone sialoprotein (IBSP) gene expression levels in mesenchymal stem cells exposed to the ionic dissolution products from customized bioglass particles according to various embodiments of the present invention;

FIG. 11 is a graph showing dissolution data for customized bioglass particles according to embodiments of the present invention including Copper ions; and

FIG. 12 is a graph showing dissolution data for customized bioglass particles according to embodiments of the present invention including Magnesium ions.

### DETAILED DESCRIPTION OF THE INVENTION

Disclosed herein are bioactive ceramics (e.g., bioglasses) that are customized so that their ion release profile matches the bone healing process (e.g., post-fracture), and methods for forming same. Also disclosed herein are implants containing or formed from such bioactive ceramics, and methods for forming same.

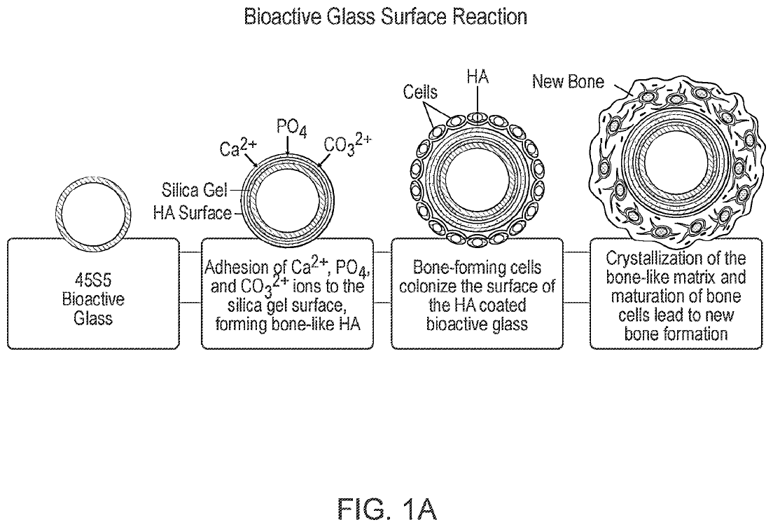

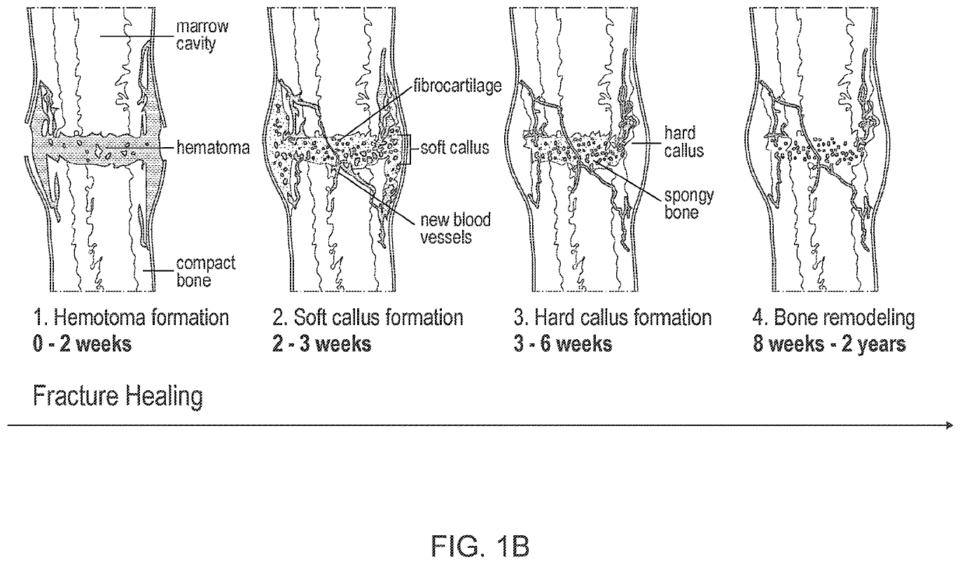

In various embodiments of the disclosed invention, a bioglass (i.e., bioglass particle) is formed in multiple layers. Each layer contains ions that play a role in a specific stage/stage of the bone healing process, which process is illustrated in FIG. 1B. In Step or Stage (used interchangeably herein) 1 of the bone healing process, a hematoma forms within 0-2 weeks after the bone fracture. Stage 2 is soft callous formation, which takes place within 2-3 weeks after the bone fracture. New blood vessels and neocartilage also form during Stage 2. Stage 3 is hard callous formation, which takes place within 3-6 weeks after the bone fracture. Spongy bone also forms during Stage 3. Stage 4 is bone remodeling, which generally takes place from 8 weeks to 2 years after the bone fracture.

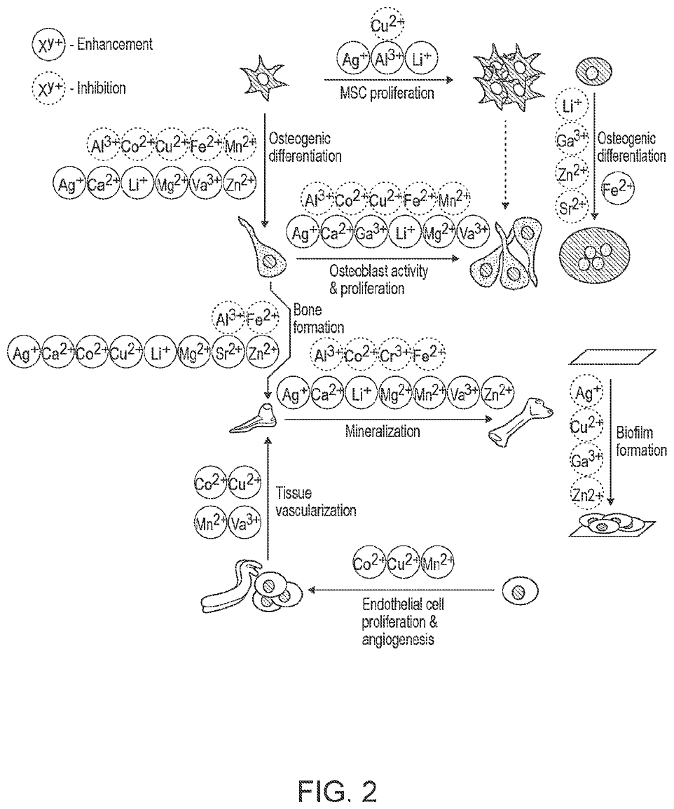

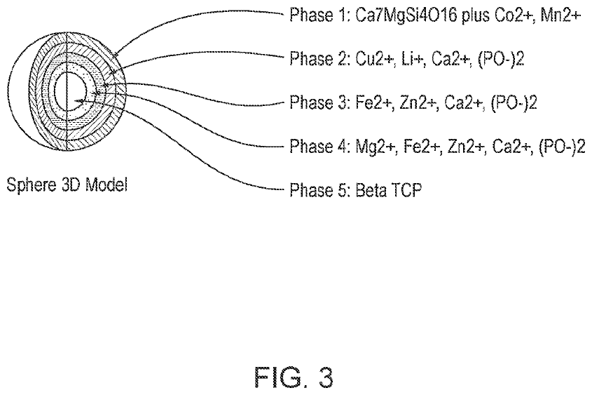

As illustrated in FIG. 2, the different stages of bone healing are associated with the activity of various ions. In various embodiments of the present invention, these ions are included in layers of customized bioglass particles. One such embodiment is illustrated in FIG. 3, wherein the bioglass particle is formed as a multi-layered sphere, and the respective layers of the sphere include the following ions, which correspond to the stages of bone healing discussed above:

| Stage of | Bioglass layer and Ions included |
|---|---|
| Bone Healing | therein, and their actions |
| 1 | 1^{st} (outermost layer) |
| Hematoma | Bredigite Ca7MgSi4O16, which show high |
| formation | angiogenic and osteogenic activity |
| | Also Cu2+, Co2+, Mn2+, which initiate/kickstart |
| | angiogenic activity |
| 2 | 2^{nd} |
| Soft callous | Start mesenchymal stem cell (MSC) Osteoblast |
| formation | proliferation with Cu2+ Li+, Ca2+, (PO−)2 |
| 3 | 3^{rd} |
| Hard callous | Start MSC differentiation and mineralization |
| formation | with Fe2+, Zn2+, Ca2+, (PO−)2 |
| 4 | 4^{th} (innermost) |
| Bone | Continue mineralization with Mg2+, Fe2+, |
| remodeling | Zn2+, Ca2+, (PO−)2 |
| 5 | 5^{th} (alternative innermost) |
| Ongoing Bone | Continue mineralization with Beta |
| remodeling | tricalcium phosphate (TCP) |
| (Optional) | |

In some embodiments, the fifth layer with Beta TCP is not included in the customized bioglass particles.

In other embodiments, different groups of ions may be included in the various layers of the customized bioglass particles.

In various embodiments, after the customized bioglass particles are formed according to the present invention, the particles are formed/agglomerated into specific shapes for implantation.

In other embodiments, the customized bioglass particles are formed in other, non-spherical shapes. Such shapes may include, for example, cubic or irregular shapes. In other embodiments, the customized bioglass particles are formed in spherical shapes. In another embodiment, the bioglass is not in a layer form but contains one or more novel ions.

In various embodiments, the customized bioglass particles, or implants formed therefrom, are implanted into the bone defect area in a patient. For orthopedic applications, such as use in repairing bone fractures and other defects, in one embodiment the customized bioglass particles are deposited directly into the fracture site, wound, defect, void, etc. In other embodiments for orthopedic applications, the customized bioglass particles are deposited into a biocompatible sponge to form a composite. Further composite compositions may contain mineral (like HA), GAGs, gelatin, peptides, various types of collagen and growth factors (GFs). For spinal applications, such as spinal fusion in one embodiment the customized bioglass particles are deposited into a biocompatible sponge to form a composite. Other composite compositions may contain mineral (like HA), GAGs, gelatin, peptides, various types of collagen and GFs. The bioglass-loaded sponge is then placed on (i.e., laid across) a portion of the vertebrae/spine to be repaired. In some embodiments, the vertebrae are scored or otherwise treated to cause bleeding that promote implant integration and healing.

Once implanted into the bone defect area, the customized bioglass particles dissolve in the body, and the ions included in the respective layers are released to coincide with the respective corresponding stage/step of the bone healing process. This timed release of the ions enhances bone healing when released in concert with the bone healing process.

In another embodiment, once implanted or place on a wound, the soft tissue defect area, the customized bioglass particles dissolve in the body, and the ions included in the respective layers are released to coincide with the respective corresponding stage/step of a particular wound healing process. This timed release of the ions enhances the soft tissue wound healing process.

In various embodiments, the customized bioglass particles may be formulated or mixed with other bioactive and/or therapeutic substances prior to implantation. Such bioactive and/or therapeutic substances include, for example, blood, bone marrow aspirate, one or more polymers, hyaluronic acid, bone cement, mineral (like HA), GAGs, gelatin, peptides, various types of collagen and growth factors (GFs). Various methods of forming the customized bioglass particles will now be described. These embodiments can be utilized for both hard and soft tissue applications.

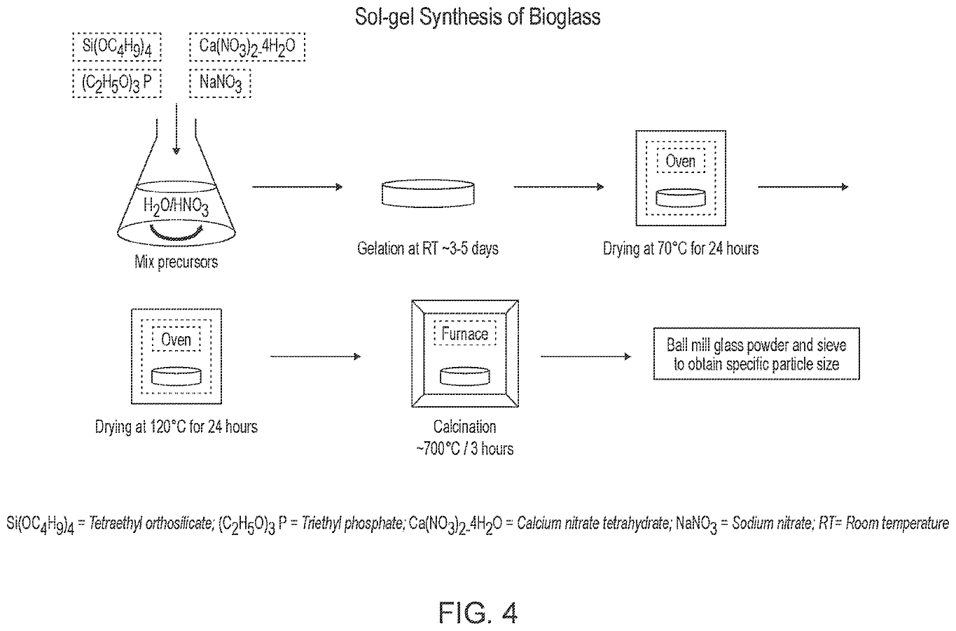

In one embodiment, the customized bioglass particles are formed via a sol-gel synthesis method. Referring now to FIG. 4, precursors (i.e., tetraethyl orthosilicate (Si(QC4H9)4), triethyl phosphate ((C2HsO)3P), calcium nitrate tetrahydrate (Ca(NQ3)2·4H2O), sodium nitrate (NaNQ3), water (H2O)) and/or nitric acid (HNQ3) are placed in a in a flask or other appropriate container, the novel ion is added, and the ions are added after the sodium nitrate and the solution is mixed for a up to 3 hours. Other precursors are possible in various embodiments. The resulting mixture undergoes gelation at room temperature for 3-5 days. The resulting gel is then aged in an oven for 24 hours at 70° C. The aged gel is then dried in an oven for 24 hours at 120° C. The dried gel then undergoes calcination by placing it in a furnace for 3 hours at 700° C. After the calcination step, the resulting customized bioglass is milled (e.g., ball milled) into a bioglass powder, and sieved to obtain specific particle size. This solution process is repeated for the next layer. In various embodiments, the particle size ranges from 1 μm-2000 μm.

The customized bioglass formed by the above sol-gel synthesis method has the following properties:
(1) Highly porous and high surface area
(2) Faster hydroxycarbonate layer formation
(3) Lower fabrication temperature leads to greater control on composition and homogeneity
(4) Batch-to-batch variations occurs because sol-gel synthesis is not a continuous process

To create layers on either the sol-gel or heat created bio-glasses, the sol-gel process was utilized. The layers are created by placing the bio-glass particles in the ion/precursor solution, subjecting the resulting mixture to gelation at room temperature, aging the resulting gel in an oven, drying the aged gel in an oven, subjecting the dried gel to calcination to form the bioactive ceramic, milling the resulting customized bioactive ceramic into a powder and sieving the powder to obtain specific particle sizes. Another process to create layers, is to place the bio-glass particles into a more dilute solution of the ion/precursor solution to grow the layer on the surface of the bio-glass particles for a period of time (time-layer thickness), filter the ion/precursor solution, aging the resulting filtrate in an oven, drying the aged gel in an oven, subjecting the dried gel to calcination to form the bioactive ceramic, milling the resulting customized bioactive ceramic into a powder and sieving the powder to obtain specific particle sizes.

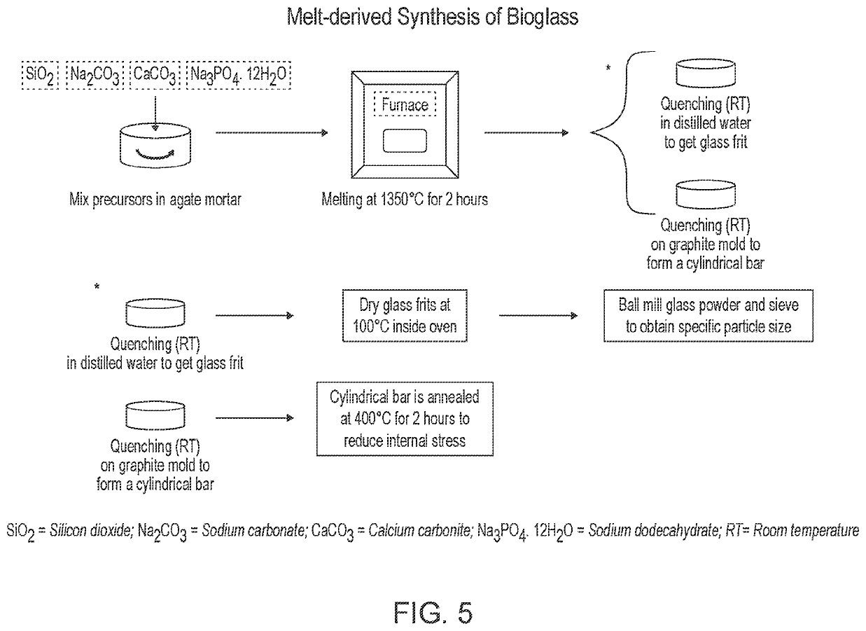

In another embodiment, the customized bioglass particles are formed via a melt-derived synthesis process. Referring now to FIG. 5, precursors (i.e., silicon dioxide (SiO2i, sodium carbonate (Na2CO3), calcium carbonate (CaCQ3), sodium phosphate dodecahydrate (Na3PQ4_12H2O), water (H2O) and/or nitric acid (HNQ3) and the novel ion are included in an agate mortar. Other precursors are possible in various embodiments. The loaded agate mortar is placed in a furnace for 2 hours at 1,350° C. to melt. The melted agate mortar is then quenched in distilled water at room temperature to obtain a customized bioglass frit. The customized bioglass frit is then dried in an oven at 100° C. The resulting customized bioglass frit is milled (e.g., ball milled) into a bioglass powder, and sieved to obtain specific particle size. In various embodiments, the particle size ranges from 1 μm-2000 μm.

Also illustrated in FIG. 5 is a variation of the melt-derived synthesis process in which the melted agate mortar is quenched on a graphite mold at room temperature to form a cylindrical bar of customized bioglass. The customized bioglass bar is then annealed for 2 hours at 400° C. in a furnace and then allowed cool within the furnace to reduce internal stress. (Annealing is performed by heating bioglass at a specific temperature and then cooling at a very slow rate).

The customized bioglass formed by the above melt-derived synthesis method has the following properties:
(1) Less porous and low surface area
(2) Dissolution rate of the customized bioglass depends on particle size and composition
(3) Finer powder exhibit higher surface area and provide more exposed surface for dissolution
(4) Finer powder degrade and resorb faster

Physical properties of the customized bioglass particles, such as porosity and surface area, influence glass dissolution and subsequent mechanisms leading to hydroxycarbonate layer formation.

In another embodiment, four distinct types of customized bioglass particles are formed, and each particle has a different thickness “dissolution sacrificial layer” that delays the release of the ions needed for the particular bone healing step. These four particle types are described below:
Particle Type 1: a particle having no sacrificial layer and that releases ions that attack (i.e., correspond to) Step 1 of the bone healing process (hematoma formation).
Particle Type 2: a particle having a thin sacrificial layer that lasts 1-2 weeks, and then bulk releases ions that attack Step 2 of the bone healing process (soft callus formation).
Particle Type 3: a particle having a thicker sacrificial layer that lasts 3 weeks, and then bulk releases ions that attack Step 3 (hard callus formation) of the bone healing process.
Particle Type 4; a particle having the thickest sacrificial layer, that lasts 6 weeks and then bulk releases ions that attack Step 4 (bone remodeling) of the bone healing process.

In one embodiment, Particles 2 and 3 release the same ions, according to the ion chart (see FIG. 2).

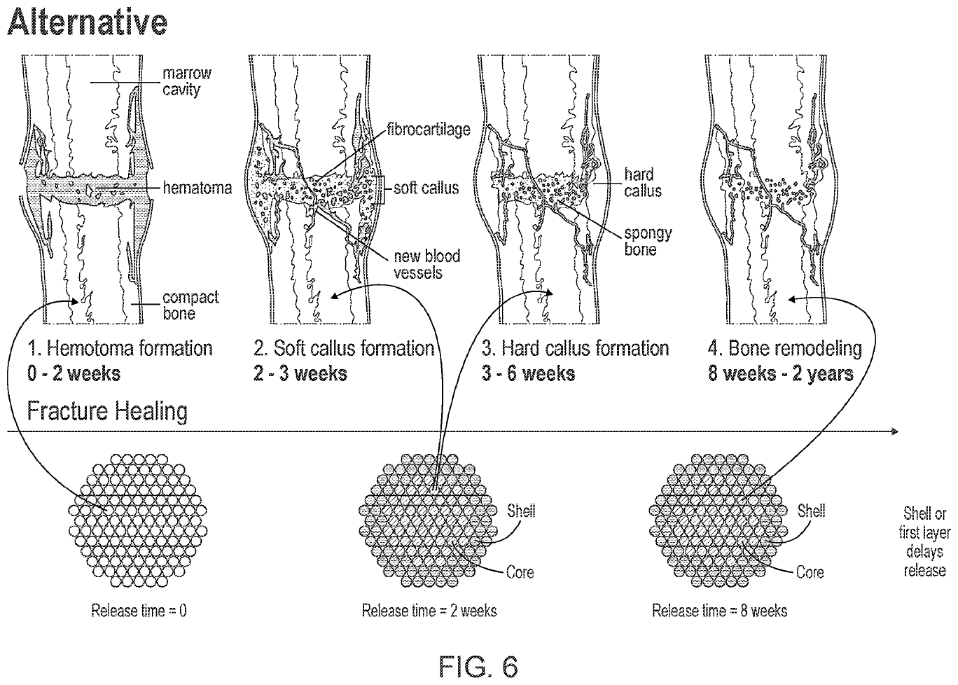

An alternate embodiment of the customized bioglass particle is illustrated in FIG. 6. Each particle has a core and a shell, or coating, which have two different formulations. In one embodiment, the shell has an ion content of 0.01-10% and release time of 3 weeks, to correspond/coincide with Steps 2 and/or 3 of the bone healing process (i.e., soft callus formation and hard callus formation, respectively), and the core has an ion content of 0.01-10% and delayed release time of 8 weeks, to correspond/coincide with Step 4 of the bone healing process (i.e., bone remodeling).

In various embodiments, each layer of the customized bioglass particle can contain more than one ion.

In other embodiments, the bioglass contains single ions, and multiple bioglasses can be used in one medical device (i.e., without layers).

In another alternate embodiment, the customized bioglass is formed in a multi-layer “sandwich formation”. In one such embodiment, the core or base layer is Mg2+ and the top and bottom (i.e., outer) layers are Cu2+.

### EXAMPLES

The below examples discuss bioglass customized with Copper and Magnesium ions. However, the bioglass of the present invention can also be customized with other ions, including, but not limited to, Zinc, Lithium and Silver.

### Example I—Effects on Customized Bioglass on Bone Healing

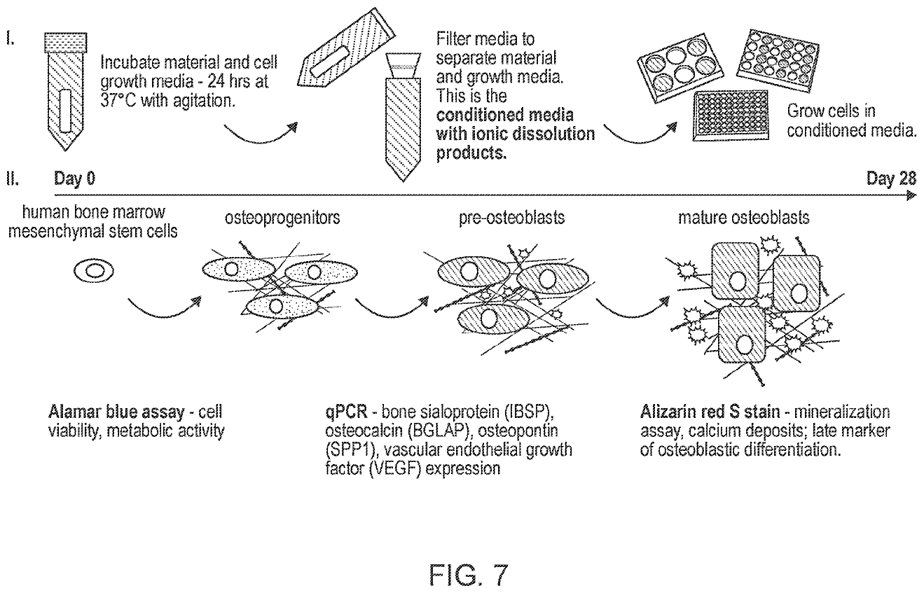

Reference is made to FIG. 7, which shows a cellular assay used to evaluate the therapeutic effects of customized bioglass particles according to various embodiments of the present invention.

As the biodegradation of 45S5 bioglass occurs, ions are released into the biological environment to control cellular functions and physiological processes such as osteogenesis and wound healing. In order to examine the effects of the customized bioglass material (i.e., 45S5 bioglass particles customized with Copper and 45S5 bioglass particles customized with Magnesium) on cellular behavior, conditioned media containing the ionic dissolution products from the customized bioglass was prepared by immersing it in cell growth media for 24 hours at 37° C. and 5% CO2. The particulates were removed by filtration through a 0.2 mm filter and media supplements (penicillin-streptomycin and 10% fetal bovine serum) was added to the filtrate or conditioned media. Human bone marrow mesenchymal stem cells were seeded at a density of approximately 1×10⁴ cells/cm² and treated with complete conditioned media containing the appropriate supplements. The media was replaced with complete conditioned media every 3 to 4 days.

Quantitative real time RT-PCR was used to examine the gene expression of various markers specific to osteogenesis. Cells were disrupted by using QIAshredder spin columns (Qiagen), and total RNA was extracted using the RNeasy Plus Mini Kit (Qiagen). From each sample, RNA was reverse transcribed into single-strand complementary DNA (cDNA) using SuperScript IV VILO Master Mix (Invitrogen). Real-time amplification was achieved on a QuantStudio 6 Flex Real-Time PCR system (Applied Biosystems) using TaqMan Fast Advance Master Mix and the TaqMan Gene Expression Assays (Applied Biosystems) for the genes of interest, normalizing the expression of each to 18S rRNA expression. The fold change in gene expression relative to the negative control was calculated using the delta delta CT method.

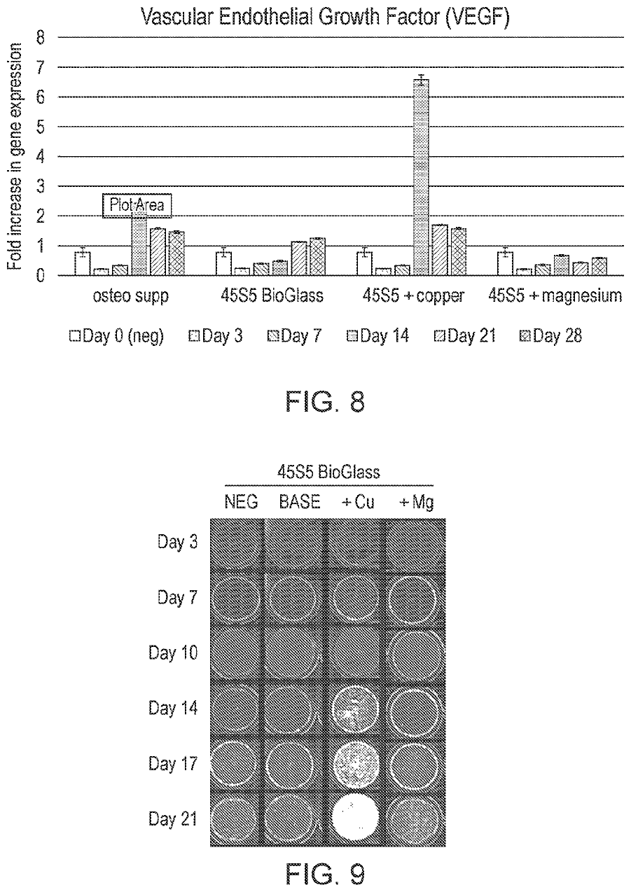

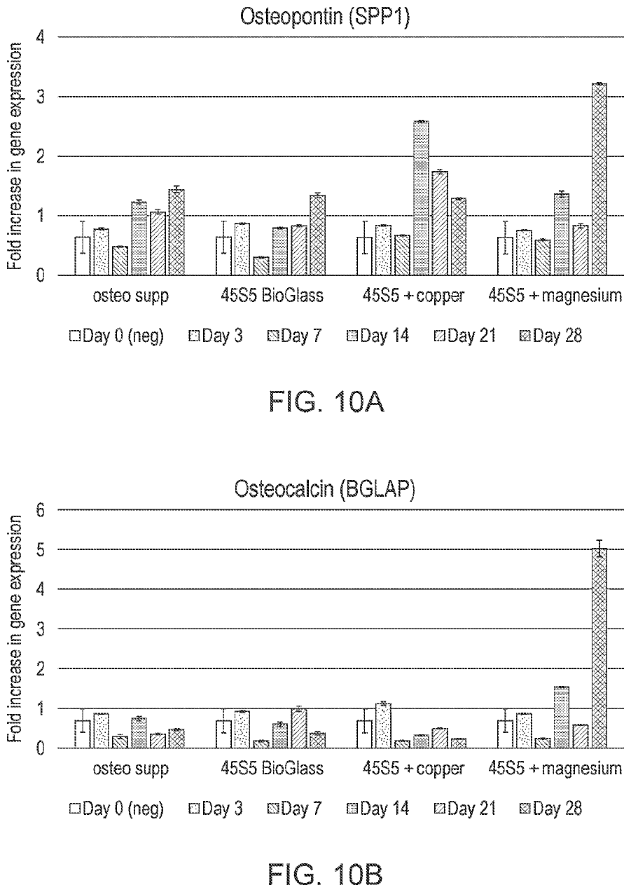

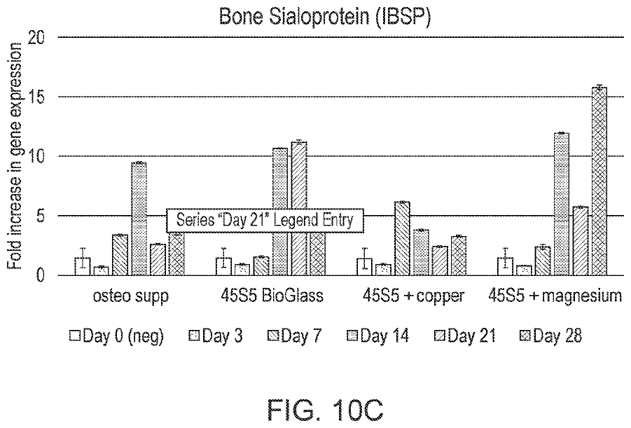

Gene expression of vascular endothelial growth factor (VEGF) was measured in the mesenchymal stem cells of the various cell populations since VEGF is a master regulator of angiogenesis during growth, development, and diseased states. As shown in FIG. 8, the cells in conditioned media from 45S5 bioglass particles customized with Copper ions generally exhibited higher VEGF gene expression than cells in conditioned media with non-customized 45S5 bioglass particles or and the osteogenic supplement (osteo supp) control, typically consisting of b-glycerophosphate, L-ascorbic acid, and dexamethasone (i.e., media without any bioglass particles), most significantly at Day 14. The higher VEGF gene expression corresponds to a dramatic increase in angiogenesis (blood vessel formation) in healing bone.

Bone regeneration is a complex and well-coordinated physiological process that involves interactions between various cells and osteogenic signals to form new mineralized tissue. To evaluate in vitro mineralization of mesenchymal stem cells in response to ionic dissolution products from customized bioglass, cultures of bone marrow-derived mesenchymal stem cells were stained with Alizarin Red Staining Solution (Sigma) to identify calcium-containing nodules. Images of the stained cells were captured on a Keyence BZ-X800 fluorescence microscope. As shown in FIG. 9, the cells in conditioned media with 45S5 bioglass particles customized with Copper ions generally exhibited a higher volume/concentration of calcium deposits/mineralization than in conditioned media with non-customized 45S5 bioglass particles or the control.

The 45S5 bioglass particles customized with Copper ions enhance the bone healing process by addressing all stages of the process, including early-stage angiogenesis/vascularization, in contrast with non-customized 45S5 bioglass particles.

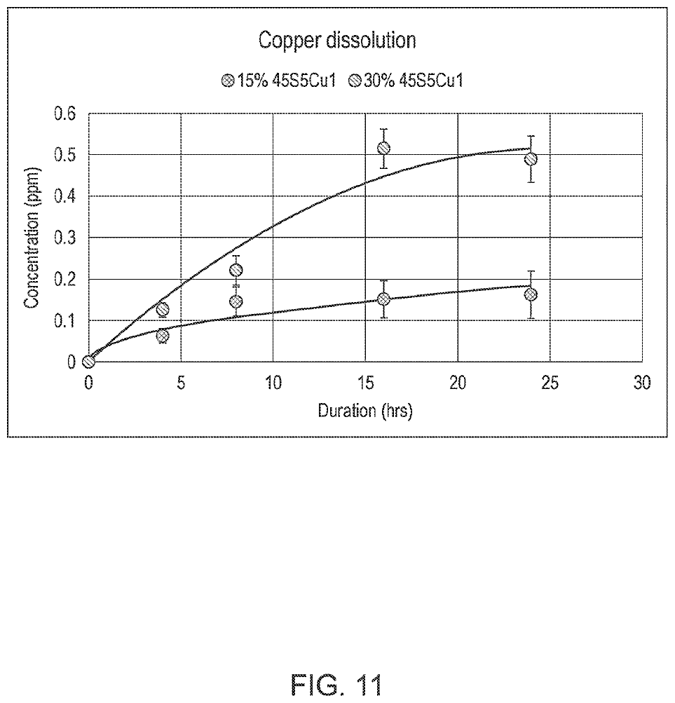

Gene expression of osteopontin (SPPI) was measured in the mesenchymal stem cells of the various cell populations since osteopontin is involved in the proliferation and migration of bone-related cells and their adhesion to hydroxyapatite, and is therefore an integral part of bone regeneration and remodeling. As shown in FIG. 11A, the cells grown in conditioned media from 45S5 bioglass particles customized with Copper and Magnesium ions generally exhibited higher osteopontin gene expression than cells in conditioned media with non-customized 45S5 bioglass particles or and the “osteo supp” control (i.e., media without any bioglass particles), most significantly at Days 14, 21 and 28.

Gene expression of osteocalcin (BGLAP) was measured in the mesenchymal stem cells of the various cell populations since osteocalcin binds to calcium, regulates osteoblast development, and acts in the bone matrix to regulate mineralization. As shown in FIG. 1 1B, the cells in conditioned media from 45S5 bioglass particles customized with Magnesium ions generally exhibited higher osteocalcin gene expression than cells in conditioned media with non-customized 45S5 bioglass particles or and the “osteo supp” control (i.e., media without any bioglass particles), most significantly at Days 14 and 28.

Gene expression of bone sialoprotein (IBSP) was measured in the mesenchymal stem cells of the various cell populations since bone sialoprotein aids in the incorporation of calcium and nodule formation by osteoblasts, and is therefore a critical regulator of bone formation and repair. As shown in FIG. 11C, the cells in conditioned media from 45S5 bioglass particles customized with Magnesium ions generally exhibited higher bone sialoprotein gene expression than cells in conditioned media with non-customized 45S5 bioglass particles or and the “osteo supp” control (i.e., media without any bioglass particles), most significantly at Day 28.

The 45S5 bioglass particles customized with Copper and Magnesium ions enhance the bone healing process by addressing all stages of the process, including early-stage angiogenesis/vascularization, late-stage healing and hard tissue formation, in contrast with non-customized 45S5 bioglass particles.

Embodiments of the customized bioglass according to the invention work in concert with the natural bone healing process via the upregulation of VEGF early in the healing process (due to the bioglass particles customized with Copper ions) and the triple action of bioglass ions, Copper ions and Magnesium ions on the hard tissue formation.

In various embodiments, the ion(s) constitute 0.1%-10% by weight of the customized bioglass, as the ion(s) replaces the calcium in traditional bioglass compositions when divalent, and sodium when the ions are monovalent.

As discussed in the example, the bioglass particles can be customized to include Copper ions, Magnesium ions or both Copper ions and Magnesium ions (e.g., at 5% each). Being customized with both Copper and Magnesium ions enhances all stages of bone healing, as discussed above.

In other embodiments, the ion(s) constitute 10% to 30% by weight of the customized bioglass.

Bioglass particles customized with Copper ions also exhibit enhanced antimicrobial action (i.e., including both pH antimicrobial action as exhibited in traditional bioglass particles, and additional antimicrobial properties of Copper ions.

### Example 2—Jon Dissolution in Customized Bioglass

FIG. 11 is a graph showing dissolution data for bioglass particles customized with Copper 10 ns. Dissolution rates for customized 1% Cu 45S5 bioglass particles that were formulated at 15% and 30% in a collagen composite matrix are shown.

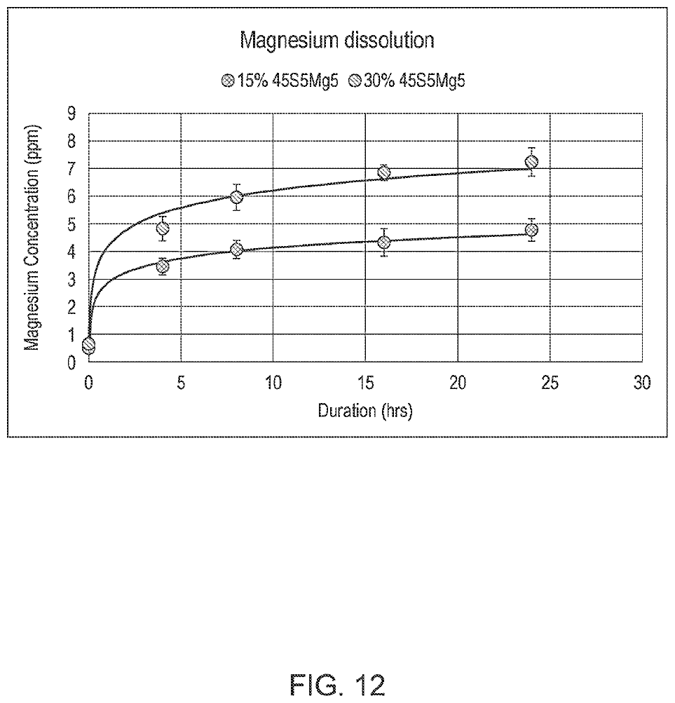

FIG. 12 is a graph showing dissolution data for bioglass particles customized with Magnesium ions. Dissolution rates for customized 5% Mg 45S5 bioglass particles that were formulated at 15% and 30% in a collagen composite matrix are shown.

While bone healing applications have been identified in connection with use of the customized bioglass particles disclosed herein, additional applications include use in spinal surgery, dental surgery and wound healing (i.e., soft tissue repair). In one embodiment, the use of silver ions in bioglass for wound healing/soft tissue repair is modified by replacing the silver ions with Copper ions. Such customized bioglasses can be used in medical devices for soft tissue repair. As tendons and ligaments have low vascularity, the angiogenesis-enhancing properties of the customized bioglass particles (i.e., with Copper) discussed above are especially advantageous for such soft tissue repair applications.

Various embodiments of the invention include medical devices that contain an ion-containing bioglass that upregulates cellular growth factors, including, but not limited to, VEGF. Such medical devices may be customized for utilization in wound healing, orthopedics, dental, cardiovascular, spine, tendon or ligament applications.

In general, any combination of disclosed features, components and methods described herein is possible. Steps of a method can be performed in any order that is physically possible.

All cited references are incorporated by reference herein.

Although embodiments have been disclosed, the invention is not limited thereby.

1. Bioactive ceramic particles, each of the particles comprising a least one ion.

2. The particles of claim 1, wherein the bioactive ceramic is a bioglass.

3. The particles of claim 2, wherein at least a portion of the bioglass is the 45S5 form of bioglass.

4. The particles of claim 2, wherein the at least one ion is in a range of 0.1%-10% by weight of one of the particles.

5. The particles of claim 1, wherein the at least one ion is selected from the group consisting of Cu2+, Mg2+, Co2+, Mn2+, Ca7MgSi4O16, Li+, Ca2+, Fe2+, Zn2+, Ag+ and (PO—)2.

6. The particles of claim 1, wherein each of the particles comprises a plurality of layers, each layer arranged to include the at least one ion.

7. The particles of claim 6, wherein each of the particles is formed as a multi-layered sphere.

8. The particles of claim 6, wherein each of the particles is formed as a multi-layered sandwich.

9. The particles of claim 8, wherein the sandwich includes a core layer having a first ion and at least two outer layers each having a second ion.

10. The particles of claim 9, wherein the first ion is Mg2+.

11. The particles of claim 9, wherein the second ion is Cu2+.

12. The particles of claim 6, wherein the at least one ion is active in a separate stage of bone healing.

13. The particles of claim 12, wherein the plurality of layers includes a first layer that includes a first group of the at least one ion that is active in a hematoma formation stage of bone healing.

14. The particles of claim 13, wherein the first group of the at least one ion includes ions selected from the group consisting of Ca7MgSi4O16, Co2+ and Mn2+.

15. The particles of claim 13, wherein the plurality of layers includes a second layer that includes a second group of the at least one ion that is active in a soft callous formation stage of bone healing.

16. The particles of claim 15, wherein the second group of the at least one ion includes ions selected from the group consisting of Cu2+, Li+, Ca2+ and (PO—)2.

17. The particles of claim 15, wherein the plurality of layers includes a third layer that includes a third group of the at least one ion that is active in a hard callous formation stage of bone healing.

18. The particles of claim 17, wherein the third group of the at least one ion includes ions selected from the group consisting of Fe2+, Zn2+, Ca2+ and (PO—)2.

19. The particles of claim 17, wherein the plurality of layers includes a fourth layer that includes a fourth group of the at least one ion that is active in a bone remodeling stage of bone healing.

20. The particles of claim 19, wherein the fourth group of the at least one ion includes ions selected from the group consisting of Mg2+, Fe2+, Zn2+, Ca2+ and (PO—)2.

21-32. (canceled)
